(19) United States
(12) Patent Application Publication
(10) Pub. No.: US 2025/0288462 A1
(43) Pub. Date: Sep. 18, 2025

(54) METHOD AND ASSEMBLY FOR RECALIBRATING THE FOCUS OF AN OPHTHALMOLOGICAL SYSTEM FOR INTRAOCULAR LASER TREATMENT

(71) Applicant: Carl Zeiss Meditec AG, Jena (DE)

(72) Inventors: Daniel BUBLITZ, Rausdorf (DE); Martin HACKER, Jena (DE); Manfred DICK, Gefell (DE); Alexander GRATZKE, Jena (DE); Stephan MERKEL, Gera (DE); Peter KLOPFLEISCH, Jena (DE); Jürgen HAESE, Kahla (DE)

(21) Appl. No.: 18/694,856

(22) PCT Filed: Sep. 22, 2022
(86) PCT No.: PCT/EP2022/076402

§ 371 Date: Nov. 18, 2024

(30) Foreign Application Priority Data
Sep. 24, 2021 (DE) 10 2021 210 661.7

(51) Int. Cl. A61F 9/008; A61B 17/00; A61B 18/20; A61B 90/00; G16H 20/40

(52) U.S. Cl. CPC A61F 9/008; G16H 20/40; A61B 2017/00725; A61B 2018/2025; A61B 2090/061; A61F 2009/00844; A61F 2009/00874

(57) ABSTRACT

A method of recalibrating the focus of an ophthalmological laser treatment system. A target laser beam is focused on at least one target structure ZS₁ in the eye to be treated by changing distance A between the laser treatment system and the eye until it is detected that the target laser beam of the laser treatment system is focused on the target structure ZS₁. For selected values of a change in distance ΔA between the laser treatment system and the eye, each focus position PF to be adopted of the laser beam in the OCDR signal profile is estimated approximately, using the parameters A₁ and PZS₁, on the basis of the distance A₁ of the position of a selected reference structure PRS, as well as on the basis of the position of the target structure PZS₁ in the OCDR signal profile, in each case relative to a reference plane RE.

### CLAIM TO PRIORITY

This application is a United States National Stage entry of Application PCT/EP2022/076402, filed Sep. 9, 2022, which claims priority to DE Patent Application No. 10 2021 210 661.7, filed Sep. 24, 2021, each of which are incorporated by reference in their entireties in this application.

### TECHNICAL FIELD

Embodiments of the invention relate to a method and an arrangement for recalibrating the focus of an ophthalmological laser treatment system which, in addition to a treatment laser unit, an imaging unit and an optical system for focusing and beam superposition, also comprises an OCDR system and a control unit. In this context, OCDR is understood to mean a one-dimensional OCT, i.e. an OCT which can record A-scans (depth profiles). The imaging system can be configured as a camera-based system or else as an observation unit for the operator's eye (“laser slit lamp”).

### BACKGROUND

According to the known prior art, there already are numerous solutions for surgical, ablative, thermal or else therapeutic laser treatment of tissues in the eye, for example of the cornea, sclera, trabecular meshwork, retina, crystalline lens or vitreous humor.

Independently of the type of treatment, i.e. the tissue to be treated, it is particularly important to observe appropriate rules during the use of a laser, to avoid injury to the eye. In addition to the laser power and the overall beam exposure, it is particularly important in this context to align the focus of the treatment laser as exactly as possible with the tissue to be treated since deviations might lead to damage to adjacent tissues of the eye or the sought-after treatment effect might not be obtained on the tissue to be treated. This is described in more detail by way of example on the basis of the treatment of the vitreous humor of the eye.

The vitreous humor consists of a usually clear, gel-like substance in the interior of the eye between the lens and the retina. In young people, the vitreous humor is largely clear and has contact with the retina. Over the course of a lifetime, the vitreous humor liquefies and increasingly detaches from the retina; this is referred to as posterior vitreous detachment. This is a normal aging process which usually occurs after the age of 50. Vitreous humor components may successively collapse in the interior of the eye and framework substances and concentrations of the vitreous humor are rendered increasingly visible to the patients. Since they may also move across the visual field, they are also referred to as “floaters”. Often, as cause of vitreous opacification, membrane-like structures are also present on the posterior side of the vitreous humor following the vitreous humor detachment, sometimes even the remains of blood should retinal injury have occurred during the vitreous humor detachment. In rare cases, vitreous opacification may also be present as crystal-like precipitates in the vitreous humor in the case of metabolic problems.

Even if vitreous opacification usually does not have a pathological cause, it is not as harmless as generally assumed because it can impair, sometimes quite significantly, the quality of life and also work productivity of the affected parties. This opacification is perceived especially against a bright background, for example when working on a computer, when reading or when looking at the blue sky or snow, and disturbs the visual faculty. Vitreous opacification that is flung into and out of the central field of vision as a result of the reading movements when reading can be particularly bothersome.

Since these often have the form of a “flying gnat”, they are described using the technical term “mouches volantes”-which comes from the French. However, the opacification may also have different shapes, for example be branch-, ring- or star-shaped, or else be present as point clouds. In the following text, the term “vitreous opacification” is used for the vitreous opacification to be treated, irrespective of its type or form.

In general, vitreous opacification does not disappear without treatment because the immune system does not recognize this as abnormal and therefore does not destroy it. However the affected parties can hardly ignore or overlook it. Certain types of vitreous opacification, such as those caused by residual blood following retinal bleeding, are partly resorbed by the body again, even if this takes weeks or months.

In what is known as vitrectomy, the vitreous humor is partly (core vitrectomy) or completely comminuted, aspirated and removed after the eye has been opened up using cutting tools. Such an intervention is carried out routinely in the case of retinal detachments or the peeling of epiretinal membranes, but is usually considered a disproportionate therapy for removing the localized vitreous opacification. Moreover, vitrectomy is invasive, requires a stay at a clinic and harbors the risks linked to surgical interventions, in particular the frequent inducement of a cataract, seldom a retinal detachment and very seldom, but possible, endophthalmitis.

So-called laser vitreolysis now offers a low-risk treatment alternative. Laser vitreolysis is a sparing, low-risk and pain-free laser treatment, by application of which the vitreous opacification can be vaporized or atomized without opening up the eye.

In the case of laser vitreolysis, short laser light pulses are directed at the vitreous opacification in order to obtain optical breakdown or photodisruption there on account of the high laser intensity in the focal region. The vitreous opacification and the vitreous humor surrounding it absorb the laser energy and a cutting or expanding laser plasma is formed, as a result of which the floaters are vaporized and/or comminuted and can as a result dissolve. The treatment causes little pain and is without risk of infection. Laser vitreolysis provides a safe method for the sparing treatment of bothersome vitreous opacification should it be possible to ensure that important and sensitive eye structures, for example the capsular bag, the crystalline lens or retinal regions, especially the macula, are not damaged by the laser.

To this end for example, it is important to be able to align the treatment laser focus as precisely as possible with the vitreous opacification to be treated and avoid an incorrect alignment of the treatment laser focus with adjacent, possibly sensitive eye structures such as the capsular bag, optic nerve head or macula. In turn, the axial treatment laser focus alignment is particularly important in this context since the vitreous opacification to be treated and the eye structures to be spared are generally located at different depths within the eye.

However, the success of the treatment depends on the type of vitreous opacification. The treatment is particularly successful in the case of so-called Weiss rings. Tissue strands can be severed and the tissue concentrations responsible for the disturbing shadows can be eradicated.

Vitreous opacification has already been treated with YAG lasers (for example as Nd: YAG at 1064 nm) for more than three decades (Brasse, K., Schmitz-Valckenberg, S., Jünemann, A. et al. Ophthalmologe (2019) 116:73. https://doi.org/10.1007/s00347-018-0782-1). However, only the most anterior region of the vitreous humor can be treated with precision and reliable targeting, even when the current high-end devices are used. These lasers are not precise enough in the deeper vitreous humor region. However, most vitreous opacification is found there as this often is the result of posterior vitreous humor detachment. YAG lasers are often used in ophthalmology for iridotomy in glaucoma diseases and for post-cataract treatment or so-called lens polishing, i.e. for removing opacification or even a post-cataract membrane on artificial lens implants as a result of cell overgrowth. Frequency-doubled YAG lasers with laser radiation in the green range (532 nm) are also used for retinal coagulation, for example in the event of bleeding or retinal detachment. YAG lasers are less frequently also used for phacoemulsification in cataract surgery, that is to say for the liquefaction of the clouded and hardened natural lens. In this case, however, this tends to be an Er: YAG laser at a wavelength of 2940 nm and with higher water or tissue absorption, which then often has to be laboriously introduced into the eye by use of an endoscopic laser introduction with a mirror guide.

According to the known prior art, there are already numerous solutions for carrying out laser surgery on the tissue of the eye, especially in the vitreous humor.

Thus, DE 10 2011 103 181 A1 describes an apparatus and a method for femtosecond laser surgery on tissue, especially in the vitreous humor of the eye. The apparatus consists of an ultrashort pulse laser with pulse lengths ranging from approximately 10 fs-1 ps, for example approx. 300 fs, pulse energies ranging from approx. 5 nJ-5 μJ, for example approx. 1-2 μJ, and pulse repetition rates of approx. 10 kHz-10 MHz, in for example 500 kHz. The laser system is coupled to a scanning system which allows the spatial variation in the focus in three dimensions. Furthermore, a beam guidance by an optical system is provided, which images the scanner mirrors for the lateral focal displacement (x, y) into the immediate vicinity of the pupil of the eye to be treated. The beam divergence can be varied in the process, in order to realize a shift of the focus position in the axial (z) direction. In addition to this therapeutic laser scanner optics system, the apparatus furthermore consists of a navigation system coupled therewith.

US 2006/195076 A1 describes a system and method for producing incisions in ocular tissue at various depths. The system and the method focus light, possibly in a pattern, on different foci situated at different depths within the ocular tissue. A plurality of foci can be created simultaneously by way of a segmented lens. Optimal incisions can be obtained by virtue of the light being focused at different depths, either successively or simultaneously, and an extended plasma column and a beam with a lengthened waist being generated. The techniques described in this case can also be used, inter alia, to perform novel ophthalmological methods or to improve existing methods, including dissection of tissue in the posterior pole, for example vitreous opacification, membranes and the retina. The document mentions that imaging methods such as OCT or ultrasound can be used to determine the position and thickness of the lens and capsular bag, in order to be able to focus the laser with greater precision. It is also mentioned that laser focusing can be implemented by way of a direct observation of a target laser (this is known), and also that the laser focusing would alternatively also be possible by way of the direct observation of OCT or ultrasound and other medical imaging modalities; this is doubtful to say the least precisely because a target laser cannot be observed directly in the OCT or ultrasound output and precisely because there is no fixed positional relationship between e.g. laser and OCT either. Accordingly, precisely no explanations are provided either as to how the laser focal position is intended to be determined by way of OCT or ultrasound or any other medical imaging method. However, it is precisely this problem that is addressed by the method, described in the present invention, for OCDR which underlies OCT.

US 2014/257257 A1 also describes a system and a method for treating target tissue in the vitreous humor of an eye, comprising a laser unit for producing a laser beam and a detector for producing an image of the target tissue. The system also contains a computer which defines a focal spot path for emulsifying the target tissue. A comparator connected to the computer then controls the laser unit in order to move the focus of the laser beam. This focus movement is carried out to treat the target tissue while deviations of the focus from a defined focus path are minimized. To attain the accuracy required for the methods described herein, the treatment is performed using a computer-controlled laser, wherein the control reference is preferably provided by an imaging detector using a technique such as optical coherence tomography (OCT). How the position of the focus of the femtosecond laser relative to the OCT is determined is not described here.

US 2015/342782 A1 likewise relates to a system and a method for using a computer-controlled laser system, for carrying out a partial vitrectomy of the vitreous humor in an eye. Operationally, an optical channel through the vitreous humor is defined first. Vitreous-like and suspended depositions (vitreous opacification) in the optical channel are then ablated and removed from the optical channel (e.g., aspirated) in some cases. In some cases, a clear liquid can be introduced into the optical channel in order to replace the ablated material and thereby establish an unimpeded transparency in the optical channel. In general, the present invention relates to systems and methods for ophthalmological laser operations. For example, the present invention relates to systems and methods for using pulsed laser beams to remove what is known as vitreous opacification. In this solution, too, an imaging unit able to create a three-dimensional image of anatomical features in the eye is used to obtain the required accuracy. To this end, equipment based on known techniques is proposed, for example Scheimpflug equipment, confocal imaging equipment, ultrasonic equipment or else imaging systems based on optical coherence tomography (OCT). However, once again, this document does not explain how the position of the laser focus is determined relative to the OCT.

US 2018/028354 A1 likewise describes a method and a system for an ophthalmological intervention in an eye. Unwanted features are identified on the basis of an image of at least a portion of the eye. The use of images recorded by a camera directed at the pupil or else the use of images showing the volume of the eye and recorded by an OCT is proposed to this end. Unwanted features in the vitreous humor cavity are considered to be instances of vitreous opacification that impair sight, for example vitreous opacification. After the vitreous opacification has been identified and localized by an image processing system, it is automatically “shot” with laser pulses following confirmation by a physician. The laser energy evaporates at least some of the vitreous-like opacity. This procedure is repeated until the opacification of the vitreous humor has been removed. The entire procedure is repeated for each instance of opacification in the vitreous humor until the liquid of the vitreous humor is considered to be sufficiently clear. The document mentions “automated targeting” with the laser but does not mention how this is implemented, especially not how this should be implemented using OCT images.

Rather than using a complicated calibration to provide a sufficient alignment of the treatment laser focus with the tissue structures to be worked on, EP 3 578 148 A1 proposes to initially use the treatment laser to implement micro-incisions in the vicinity of the working zone, detect the position of said micro-incisions and correct a possible deviation between sought-after and actual position. However, this “trial-and-error” approach burdens the eye unnecessarily (unnecessary tissue damage and light exposure to the treatment laser light) and is not universally performable either. For example, it is not possible to create any stable micro-incisions in the vicinity of the treatment zone if an eye is partly or completely filled with fluid and if opacification floating in the fluid should nevertheless be atomized by application of the treatment laser.

A method described by ELLEX (product brochure by Ellex Medical Pty Ltd.; “Tango Reflex-Laser Floater Treatment”; PB0025B; 2018; (http://www.ellex.com)) provides for the use of a pulsed nanosecond laser (YAG) in order to decompose vitreous opacification or completely remove the latter by a transition into a gas. A pilot laser beam (i.e. a target laser in the visible spectral range, for example red) is used to sight the target area (vitreous opacification) and the latter is then “shot at” using one or more therapy laser pulses. In this case, both the pilot laser beam and the therapeutic laser pulses are manually triggered by the user. Such a manual laser treatment typically consists of two individual treatments, each having a duration of 20-60 minutes.

DE 10 2019 007 147 A1 and DE 10 2019 007 148 A1 describe systems for laser vitreolysis of vitreous opacification, which enable the safe and precise atomization of vitreous opacification (“floaters”) on the basis of a combination of a treatment laser with an OCT or an OCDR system. Here, an OCDR (optical coherence domain reflectometry) system is understood to mean a system for interferometric acquisition of one-dimensional scattering profiles, while OCT (optical coherence tomography) is understood to mean 2-dimensional or 3-dimensional imaging. In both cases variants with recording sequences (i.e. film) should also be included. In the process, minimum distances to sensitive eye structures are ensured and the activation of the laser is preferably only permitted if the focus of the treatment laser and the vitreous opacification to be treated are positioned with sufficient accuracy in relation to one another.

A disadvantage of these two approaches is that the physician has to constantly combine the available, familiar 2-dimensional view of the eye with the 3-dimensional recordings of the OCT or OCDR system using their spatial imagination, which is extremely difficult in the context of the time-critical or quick interaction with the patient. This disadvantage is rectified by the solution described in the patent application DE 10 2020 212 084.6 yet to be published.

The use of the laser energy within the scope of laser vitreolysis is non-invasive and avoids the disadvantages of surgical interventions which open up the eye, but is also linked to disadvantages and risks.

For example, targeting the laser may be difficult. Since the physician observes the vitreous humor along the beam path, it may be difficult to determine the depth of the position of the retina, the depth of the opacification of the vitreous humor or other relevant features. As a consequence, there is the risk of the opacification of the vitreous humor being missed and/or the eye being injured.

For example, the treatment of largely transparent vitreous opacification, which changes its position and is difficult to recognize and also, as a phase object, is able to create bothersome shadows on the retina, was found to be difficult.

The application of laser energy may also lead to an additional movement of the opacification of the vitreous humor, making the treatment even more difficult. Thus, the physician may need to realign the laser after each application of laser energy. This may require much time. Therefore, a treatment with the laser is complicated and causes stress, both for the patient and for the physician.

A further possible problem relates to incomplete vitreous humor detachment, which may lead to local vitreous traction right up to retinal detachment. Laser treatment in the vitreous humor can lead to changes in the balance of forces in the vitreous body due to shockwaves propagating as a consequence of said treatment, and thereby for example cause tension on the retina.

Lastly, the treatment of the vitreous opacification situated in the vicinity of sensitive structures of the eye was also found to be particularly difficult. It is not only the mechanical and thermal load but also the laser radiation itself which may lead here to damage to the retina, crystalline lens or macula, and which must be suitably limited in respect of its intensity and/or energy.

However, as to the aforementioned problems, it is incredibly important to have available the most accurate knowledge with regard to the respective current position of the treatment laser focus in relation to eye structures. For post-cataract treatment or retinal coagulation, the conventional choice in this respect has usually been to use a target laser aligned with the respective tissue structure to be treated by virtue of the operator observing the backscatter from the target laser radiation. However, this is virtually impossible if the structures to be treated are very weakly scattering objects for example, which really can only still be detected by application of highly sensitive systems such as OCT or OCDR. For example, conventional OCT and OCDR systems may have a sensitivity of more than 85, 90, 100 or 110 dB, wherein a detection of the normal vitreous humor backscatter, i.e. the sensing of vitreous opacification as well, becomes possible above approx. 90 dB.

However, if it is not target lasers but OCT-based systems that are used, then there is the complex problem of a sufficiently accurate assignment of the treatment laser focal position to a position in the OCT or OCDR scan. This is due to the fact that the axial treatment laser focal position may already change significantly in the beam path as a result of varying refractive powers (for example surface curvatures), while the latter have virtually no influence within the scope of OCT or OCDR on the positions of signals of scattering objects in the OCT or OCDR. YAG focus diameters and also axial Rayleigh lengths in the interior of the eye are of the order of approx. 20 μm, and so small deviations are already problematic if delicate objects with similar spatial dimensions are intended to be worked on. In this respect, even small changes, for example as a consequence of thermal effects in the laser during relatively long operation, may have a relevant influence and require a recalibration of the focal position in relation to the OCDR.

Some of the solutions known according to the prior art that use OCT systems assume known focal lengths, refractive powers, distance parameters and sample positions, with the result that a calculation of the focal position in relation to the OCT or OCDR would be possible in principle. However, this would still require sufficiently accurate knowledge, or highly accurate determination, of these parameters, but this would be incredibly complicated, for example in view of the determination of all relevant refractive powers (for example of the corneal and lens surfaces) in the eye. Additionally, data obtained preoperatively may be insufficient because contact pressure-dependent changes of the corneal refractive powers may arise, for example during use, for example during the use of hand-held contact glasses. Additionally, there is a significant variation in human eye anatomies. For example, eye lengths range between 14 and 40 mm and there is great variation in the depth of the anterior chambers (1.5 to 4 mm) or lens thicknesses (crystalline lenses: ˜3-4 mm, IOLs in part thinner or in part thicker, if multi-part IOLs, for example, are used).

In addition to the different focal depths and the varying refractive powers of the individual eyes, tolerances of the optics of the laser system may also lead to additional deviations in the focal position of the treatment laser.

The concept proposed in DE 10 2019 007 147 A1 of jointly varying (i.e. tuning) the focal position of OCDR and treatment laser and of deriving the calibration between OCDR and focus of the treatment laser from the resultant OCDR signal variations also requires a sufficiently finely adjustable focal position with a simultaneous, synchronized OCDR detection and, in particular, would also be slightly more complicated if different axial focal positions of OCDR and treatment laser are intended to be used (i.e. if an offset between the foci should be present). Additionally, this approach is made more difficult by virtue of the OCDR or OCT recordings having “speckle” on account of their coherent properties, and hence the point of greatest signal around the focus can sometimes only be determined with slight additional outlay (for example by a speckle suppression, as in U.S. Pat. No. 8,085,408 B2 or DE 10 2008 051 272 A1).

Therefore, it is particularly important in the case of OCDR or OCT-assisted systems to enable the focal position of the treatment laser to be estimated as accurately as possible in relation to the tissue to be treated and the eye structures to be spared. In this context, the calibrations carried out to date in accordance with the solutions from the prior art, for example using a test eye or assumed or predetermined parameters, were found to be insufficient or very complicated.

The present invention is therefore based on the object of developing a solution for an ophthalmological system for intraocular laser treatment which rectifies the disadvantages of the known technical solutions by virtue of taking account of the individuality of an eye to be treated and the tolerances of the optical system by recalibrating the focus of the treatment laser. For example, this should also render it possible to calibrate the focal position of the treatment laser vis-à-vis OCT or OCDR scans for each new treatment situation, to be precise for example following the change of a patient and/or contact glasses, or else a modification of the focal length of the treatment laser, of the accommodation state of the patient and/or of the sample spacing.

Moreover, the solution should be easy to implement and economically cost-effective, and enable a simpler, faster, and, above all, safer laser treatment on the eye.

This object is achieved by the method according to the invention for recalibrating the focus of an ophthalmological system for intraocular laser treatment comprising an OCDR system and a control unit and comprising a treatment laser unit, an imaging unit and an optical system for focusing and beam superposition, by virtue of the fact that a target laser beam of the laser treatment system is focused on at least one target structure ZS₁ in the eye to be treated by virtue of the distance A of the laser treatment system from the eye being changed until focusing of the target laser beam of the laser treatment system on the target structure ZS₁ is detected, in that there is determination of a distance A₁ from the position of a chosen reference structure PRS, and the position of the target structure PZS₁ in the OCDR signal profile, in each case in relation to a reference plane RE, and in that, for any selectable values of a change in distance ΔA of the laser treatment system from the eye, the respective assumable focus position PF of the laser beam of the laser treatment system in the OCDR signal profile is estimated approximately using the parameters A₁ and PZS₁.

According to a first example configuration, the laser beam of the laser treatment system (or a substitute for the laser beam, such as a target laser beam or an attenuated treatment laser beam) is focused on at least one first target structure ZS₁ in the eye to be treated by virtue of the distance A of the laser treatment system from the eye being changed between a reference plane RE at a distance ΔE in front of a plane of reference BE of the laser system (for example the front lens vertex) and a reference structure RS on the eye (for example the corneal vertex) until, at a position A₁, the focusing of the laser beam on the target structure ZS₁ is identifiable, for example on the basis of the strongest backscatter of the laser beam of the laser treatment system or of the target beam from the target structure; the (corresponding) position PZS₁ of this target structure is determined in the OCDR signal; upon focusing on further target structures ZSₙ (n=2, . . . , N), the respective change in distance ΔAₙ, required to this end, in relation to the position A₁ of focusing on the first target structure ZS₁ is determined; for each instance of focusing on one of the target structures ZSₙ, the respective position PZSₙ thereof in the OCDR is determined; and for other values of a change in distance ΔA of the laser treatment system from the eye, selectable as desired, the respective assumable focus position PF of the laser beam of the laser treatment system is determined approximately from the function

$$PF ( Δ A ) = {f}_{{A}_{1} , Δ {A}_{2} … Δ {A}_{N} , {PZS}_{1} … {PZS}_{N}} ( Δ A ) \qquad ( 1 )$$

In this context, “values selectable as desired” means distance states between eye and laser system realizable in practice. For example, the front and back side of the lens in phakic eyes and the IOL front or back side or else, to certain extent, an IOL haptic (preferably an extensive plastic haptic) in aphakic eyes are suitable as target structures.

In general, front and back side of the contact glass or cornea and the retinal surface are also suitable target structures. If vitreous opacification scatters the target laser beam strongly enough, it is also suitable as a target structure. In general, it is advantageous if use is made of target structures located as “close” as possible to the worked-on region (axially, but also laterally) in order to obtain the greatest possible accuracy for the assumed focus position PF (AA), for example with a proximity of less than 2 mm, but preferably of less than 1 mm, and even more preferably of less than 100 μm.

According to the invention, the deviation of the approximate determination of the assumable focus position PF of the laser beam from the actual position is less than 2 Rayleigh lengths of the laser focus, for example less than 1 Rayleigh length or for example less than 0.5 of the Rayleigh length. The Rayleigh length is defined as Z_{R}=n*π*w₀²/M², where n corresponds to the refractive index of the medium, w₀ corresponds to the radius of the laser beam at the focus and 1/M² corresponds to the beam quality (ideally, M=1). In this context, twice the Rayleigh length corresponds to the conventional definition of the depth of field for material processing lasers (A. Barz, H. Müller, J. Bliedtner, “Lasermaterialbearbeitung”, Hanser Fachbuchverlag; https://www.hanser-fachbuch.de/buch/Lasermaterialbearbeitung/9783446421684) and, in the case of laser applications on the eye, is for example between 9 μm (more likely for surface-near application with a large numerical aperture) and 1 mm. In principle, it is also possible to use the method for a multi-segment treatment laser focus as known from the prior art. However, this case would require an individual calibration of all focus segments by way of a switchable or multi-segment target laser or at least an individual calibration of selected treatment laser focus segments such that, in that case, the recalibration of the selected focus segment can be used as a basis for realizing a positional estimate for the other focus segments.

The dependent claims relate to preferred developments and configurations.

According to a second configuration, the point of intersection of target lasers rather than the laser beam of the laser treatment system is focused on at least one target structure ZS in the eye to be treated. For example, a continuous wave laser beam with the same or similar focal position as the laser beam of the laser treatment system is used as target laser. In this case, the focusing of the target laser on the individual target structures ZSₙ can be detected by detecting the maximum backscatter of the target laser radiation from the respective “focused-on” target structure by way of the operator's eye or by way of a camera with image processing, the latter especially if the intention is to use non-visible target laser radiation, for example at 800 or 1060 nm in the NIR.

In the proposed method, the lens back side (crystalline lens or IOL), the capsular bag back side, the retinal surface or other structures of the eye serve as target structures ZSₙ.

According to a third configuration, at least one target structure ZS with at least short-term stability is created by the laser beam of the laser treatment system or else by the target laser, for example by a laser shot from the laser treatment system or else by a modulated target laser, by virtue of bringing about at least one modification in the eye whose detectability is sufficient for a calibration and which brings about a characteristic, measurable signal change in the OCDR.

For example, in this case, the modulation of the target laser is implemented in acousto-optic, electro-optic or interferometric fashion, or there is wavelength or polarization modulation. However, it is also possible to modulate the target laser by way of, for example, current modulation or modifiable attenuators, such as a filter wheel, a chopper or the like. A characteristic laser modulation impressed therewith can then be detected again in the speckle variations by subjecting the OCDR signal to filtering matched to this modulation, in order to determine the position of the laser focus in the OCDR signal very accurately.

In an alternative, it is for example also possible that attenuated shots of the laser treatment system create gas bubbles that are detectable at least in the short term in the OCDR (they are detectable until they rise out of the OCDR beam) as a result of plasma expansion, or else a modulated target laser beam could bring about a locally modulated phase modulation or a speckle variation in the OCDR or OCT signal in the focusing region as a result of light absorption and local heating. For the latter, it is advantageous if the target laser crosses the OCDR or OCT beam in the focus region at an angle, in order to create a maximally transient signal of the target structure there. However, the modulation frequency must not be too high in this context, so that the heat dissipation still allows temperature modulations. Possible modulation frequencies range between 0.1 and 100 Hz, and for example ones between 5 and 50 Hz. Example target laser wavelengths are in the NIR, for example between 1.2 and 1.7 μm, in another example_1.3 μm and 1.5 μm, at which water is absorbed well and the light exposure of the patient's retina is low.

In comparison with the prior art, this approach renders possible focus calibrations that are repeatable as often as desired, even in unstable media without natural target structures, for example in the aqueous humor of the anterior chamber or else in a partly liquefied vitreous humor or in a saline solution replacing the latter following a vitrectomy.

In this context, it is advantageous for example that the target structure ZS created by a laser shot of the laser beam of the laser treatment system can be used not only to determine the focal positions but also to titrate the laser power.

According to a fourth configuration, the front or back side of a contact glass KG present, a technical structure situated in the contact glass or else natural or artificial eye structures, for example the front or back side of the cornea, crystalline lens or IOL, capsular bag or the retinal surface, serve as reference structure RS for determining the respective distances A between eye and laser system.

In this case, the reference structure RS realized in the contact glass KG creates a characteristic signal in the OCDR, with the realized reference structure RS for example being modifiable, for example, switchable or modulable.

According to a further example fifth configuration, the laser beam of the laser treatment system or the point of intersection of target lasers is focused on a number N of target structures ZSₙ in the eye to be treated in order to estimate the respective assumable focus position PF of the laser beam of the laser treatment system as accurately as possible from function (1) for any selectable values of a change in distance ΔA. The parameters specified as index of the function are as follows: A₁, ΔAₙ=2 . . . . N are the eye distances and their changes in relation to A₁, in the case of which there is respective focusing on the target structures ZSₙ; and PZSₙ are the target structure signal positions in the OCDR ascertained therefor in each case. ΔA₁ is no longer listed as a parameter here since A₁ is the distance of reference for all changes in distance ΔA, and hence ΔA₁=0 in fact applies. If a different distance of reference to A₁ were to be chosen, then, rather than A₁ as parameter, its distance ΔA₁ from the distance of reference could naturally also be used instead of A₁ as parameter of the function. For the function

f_{A}_{₁}_{,ΔA}_{₂}_{. . . ΔA}_{_{N}}_{,PZS}_{₁}_{. . . PZS}_{_{N}}(ΔA)

a polynomial of first to N-th degree (or a different non-linear function with N degrees of freedom, for example a Fourier series) is used by preference to determine the focus positions PF (ΔA), said polynomial being chosen so that it runs through the points ΔA_{n=1 . . . N}, PZS_{n=1 . . . N} with deviations that are as small as possible. For example, this can be implemented by fitting the polynomial or the non-linear function to the points, for example by applying the method of least squares.

However, the function (1) for determining the focus positions FP can also be a polynomial or non-linear function of a degree greater than N, by virtue of additional parameters of the contact glass or the eye being used to determine the polynomial or the non-linear function.

According to a sixth configuration, the imaging system is focused incrementally on the target structures ZSₙ together with the laser beam of the laser treatment system or the target laser, wherefore an autofocus system is used. For example, a numerical aperture (NA) of the imaging system and a numerical aperture of the laser beam of the laser treatment system differ by a factor of less than 2 in this case. In this case, it is for example if wavelengths of the imaging system and of the laser beam of the laser treatment system deviate by no more than 10% from one another.

According to a seventh configuration, the method of recalibrating the focus of a treatment system serves for laser vitreolysis, which, in addition to a focusing unit, also comprises an OCDR system and a control unit.

In this case, the laser beam of the laser treatment system is focused on two or more target structures ZSₙ (n=2, . . . , N) in the eye to be treated by virtue of, following the focusing on the first target structure ZS₁, the distance A of the treatment system for laser vitreolysis being changed by changes ΔA in relation to the eye proceeding from a reference plane RE until the focusing of the laser beam on the respective target structure ZSₙ is identifiable on the basis of the maximized backscatter of the treatment laser or target beam laser; and the change in distance ΔAₙ present is determined for this focusing situation; for this focusing situation, the position PZSₙ of the target structure ZSₙ, on which focusing is just implemented at ΔAₙ, is determined from the OCDR signal; and for other values of a change in distance ΔA, selectable as desired, the respective assumable focus position PF of the laser beam of the treatment system for laser vitreolysis is determined approximately from function (1). By preference, a respective structure in the front and in the back eye region, for example the lens back side and the retinal surface, are chosen as target structures ZSₙ.

The approximate determination of positions of the treatment laser focus

PF(ΔA)=f_{A}_{₁}_{,ΔA}_{₂}_{. . . ΔA}_{_{N}}_{,PZS}_{₁}_{. . . PZS}_{_{N}}(ΔA)

for any desired distances ΔA of the laser system from the eye is used in that case, for example, to establish the treatment laser focus position (at the time of triggering the laser shot) relative to the blocked zones of a treatment system for vitreous opacification and an activation or deactivation of the therapy laser resulting therefrom. In this context, it is also possible to detect eye movements, for example axial eye movements, for example by application of OCDR, and to then take these into account correctively when estimating a treatment laser focus position that is afflicted by a latency time.

In another example embodiment, at least one target structure ZS is chosen in the vicinity of an eye structure to be worked on. This once again also gives rise to the option of generating a permanent or transient target structure using the (attenuated) treatment laser or using the target beam laser.

In the case of treatment systems for laser vitreolysis, it is necessary to ensure that a sufficiently large pupil diameter is present in each case, depending on the working depth, in order to ensure a sufficiently short depth of field of the treatment laser. To ensure this, an imaging system for the pupil region is used by preference, with the aid of which the currently present pupil diameter is determined. In an alternative, it is also possible to use the backscatter of a target laser corresponding to the treatment laser beam at the edge of an insufficiently dilated pupil as a criterion. Example pupil diameters are >4 mm for a laser vitreolysis treatment in the anterior region, >5 mm for such a treatment in the central region and >6 mm for such a treatment in the posterior region. The treatment system prevents the treatment laser from being triggered and provides an alert if these conditions have not been satisfied. Optionally, the ambient light in the treatment room must be reduced or pupil dilation must be brought about by medication in such a case.

The method according to the invention for recalibrating the focus of an ophthalmological system for intraocular laser treatment is provided for example for realizing a treatment system for laser vitreolysis.

Possible other intraocular application options that would profit from the proposed method for recalibrating the focus of a laser treatment system for example include, without this claiming completeness however, the following:
retinal coagulation
SRT (selective retina therapy)
vitreous humor incisions, for example for treating vitreomacular traction
laser trabeculoplasty (such as SLT) or trabeculotomy (such as ALT)
post-cataract treatments
femtosecond cataract surgery (rhexis and lens incisions) and
incisions or ablations on the cornea.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

The invention is described in more detail below on the basis of exemplary embodiments. In this regard:

FIG. 1: depicts the treatment system for laser vitreolysis focused on a first target structure ZS₁ in the eye to be treated,

FIG. 2: depicts the treatment system for laser vitreolysis focused on a second target structure ZS₂ in the eye to be treated,

FIG. 3: depicts a treatment system for laser vitreolysis, which uses a contact glass with a reference structure RS, and

FIG. 4: depicts the function PF(ΔA) for determining any desired focus positions PF of the laser beam of the laser treatment system.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

An example method for recalibrating the focus of an ophthalmological system for intraocular laser treatment comprises a treatment laser unit, an imaging unit and an optical system for focusing and beam superposition, and also an OCDR system and a control unit.

According to an example embodiment of the invention, the laser beam of the laser treatment system is focused on at least one first target structure ZS₁ in the eye to be treated, by virtue of the distance A₁ of the laser treatment system from the eye being changed until focusing of the laser beam on the target structure ZS₁ is achieved. The distance A₁ is determined from the position of a chosen reference structure PRS and the position of the target structure PZS₁ in the OCDR signal profile, in relation to a reference plane RE. For any selectable values of a change in distance ΔA of the laser treatment system from the eye, it is possible to use the parameters A₁ and PZS₁ to provide an approximate estimate for the respective assumable focus position PF of the laser beam of the laser treatment system in the OCDR signal profile.

In this case, focusing of the target laser on the target structure ZS₁ can be detected by detecting the maximum backscatter of the target laser radiation from the “focused-on” target structure ZS₁ using the operator's eye or using a camera with image processing. The position of the OCDR signal PZS₁ of the target structure ZS₁ is determined thereafter.

For example, the ascertained values A₁ and PZS₁ are used to determine a function

PF(ΔA)=f_{A}_{₁}_{,PZS}_{₁}(ΔA)

which, for any selectable values of a change in distance ΔA of the laser treatment system from the eye, approximately calculates the respective assumable focus position PF of the laser beam of the laser treatment system in the OCDR signal profile.

If further target structures Z₂ to Z_{N} should be “focused on” in the same way for the purpose of increasing the calibration accuracy over large depth ranges, then the changes in distance ΔA₂ to ΔA_{N} for which the laser beam of the laser treatment system or the target laser focuses on these target structures to the best possible extent in each case are in each case set to this end, as are the positions of the target structure signals PZS₂ to PZS_{N} present in the process. Then, the values A₁ and PZS₁ and optionally ΔA₂ to ΔA_{N} and PZS₂ to PZS_{N} determined thus can be used to determine the function PF (ΔA)=f_{A}_{₁}_{,ΔA}_{₂}_{. . . ΔA}_{_{N}}_{, PZS}_{₁}_{. . . PZS}_{_{N}}(ΔA) which, for other values of a change in distance ΔA, selectable as desired, approximately determines the respective assumable focus position PF of the laser beam of the laser treatment system.

As already mentioned above, the parameters specified as index are A₁, ΔAₙ=2 . . . . N as the eye distances and their change in relation to A₁, in the case of which there is respective focusing on the target structures ZSₙ; and PZSₙ as the target structure signal positions in the OCDR ascertained therefor in each case. To determine the focus positions for function (1)

PF(ΔA)=f_{A}_{₁}_{,ΔA}_{₂}_{. . . ΔA}_{_{N}}_{,PZS}_{₁}_{. . . PZS}_{_{N}}(ΔA)

use is for example made of a polynomial of first to N-th degree or a different non-linear function with N degrees of freedom, which is chosen so that it runs through the points ΔA_{n=1 . . . N}, PZS_{n=1 . . . N} with deviations that are as small as possible. For example, this can be implemented by fitting the polynomial or the non-linear function to the points, for example by applying the method of least squares.

By preference, focusing of the laser beam of the laser treatment system on at least one target structure ZS in the eye to be treated by the treatment laser is implemented with reduced pulse energy in order to avoid photodisruption. It is also possible that an additional laser beam whose parameter does not allow permanent tissue change in the eye is used as target laser beam.

According to the invention, the deviation of the approximate determination of the assumable focus position PF of the laser beam from the actual position is less than 2 Rayleigh lengths of the laser focus, for example less than 1 Rayleigh length or in another example less than 0.5 of the Rayleigh length.

Should the laser treatment system comprise a plurality of target lasers, the point of intersection of target lasers rather than the laser beam of the laser treatment system can be focused on at least one target structure ZS in the eye to be treated. It can indicate the position of the focus of the laser beam of the laser treatment system in continuous or quasi-continuous fashion, or optionally in pulsed fashion. To this end, use can be made of a target beam laser in the visible spectral range, but it may also be in the NIR, for example, if a camera system is used. To indicate the location of the focus of the laser beam of the laser treatment system, the target laser may for example be collinearly superimposed on the laser beam of the laser treatment system and have an identical focal position. If this is subsequently used to focus on a target structure, then this is identifiable on the basis of a minimally dimensioned and maximally intensive back-scattering target laser spot on the target structure (detectable by eye in the VIS or by camera in the NIR). In an alternative, use can also be made of a plurality of target laser beams, which intersect at the location of the focus of the laser beam of the laser treatment system. In a further alternative, use can be made of one or more moving, for example rotating, target laser beams, each of which run through the location of the focus of the laser beam of the laser treatment system.

In this case, the detection of focusing on a target structure is implemented by visual or automated establishment of one or more of the following states:
maximized backscatter of the target laser from the target structure,
minimized diameter of the light distribution of the target laser beam on the target structure or
characteristic state or change in the OCDR signal of the target structure.

According to the invention, the point of intersection of the target lasers is for example focused on the target structure ZS either by minimizing the spacing of a plurality of target laser beams by the operator or a camera system or else by maximizing the backscatter from at least one target laser beam focus by way of confocal detection. In this case, the target laser for example operates in the visible spectral range or in the NIR range.

According to an example configuration, a continuous wave laser with the same or similar focal position as the laser beam of the laser treatment system is used as target laser, or else the latter is provided by a continuous wave laser with a known or calibrated limited deviation of the focal position from the laser beam of the laser treatment system, which is taken into account during laser vitreolysis. By way of example, this can then be given consideration by way of an appropriately adapted indication of the focal position or else by instantaneous focal shifts of the laser beam of the laser treatment system prior to the laser being triggered, for example by way of a change in the beam divergence realized optomechanically (for example by way of displaceable lenses) or electro-optically (for example by way of liquid crystal lenses).

By preference, the posterior side of the lens (for example an intraocular lens, IOL) or capsular bag, the anterior retinal surface or other structures of the eye serve as target structure ZS.

However, it is also possible for the target structure ZS to be created by the laser beam of the laser treatment system itself. For example, the target structure ZS created by a laser shot of the laser beam of the laser treatment system brings about a change in the vitreous humor, which in turn causes a signal change in the OCDR, for example modified backscatter or a speckle grain modification.

In this case, the target structure ZS created by a laser shot of the laser beam of the laser treatment system is temporary or modifiable, for example a gas bubble created by at least one laser shot or a speckle structure in the OCDR that has been temporarily modified as a consequence of heating. Such changes can also be created by use of the target laser beam, for example if the latter is modulated and, by way of light absorption, consequently generates a local characteristic signal fluctuation (for example a speckle variation) in the OCDR depth profile where it intersects the OCDR beam.

However, an advantage of creating a target structure ZS by a laser shot of the laser beam of the laser treatment system is that said target structure can be used not only to determine the focal positions but also to titrate the laser power. Although this titration would also be possible indirectly by way of the absorption behavior of the target laser, it would be more difficult since possible wavelength differences would have to be taken into account or avoided.

According to the invention, the front or back side of a contact glass KG present, a technical structure situated in the contact glass, or else eye structures such as the front or back side of cornea, lens or capsular bag or the retinal surface are used as reference structure RS for the OCDR system.

If the ophthalmological system for intraocular laser treatment requires a contact glass KG, the technical structure realized as reference structure RS can for example be designed such that a characteristic signal in the OCDR is created, for example a signal having a specific signal level, plateau, curve, position, distance or multiple peaks, or a characteristic polarization dependence. For example, the reference structure RS realized in the contact glass KG could be modifiable, for example switchable or modulable, for example by way of a modification of the scattering or polarization. For example, this could be realized by way of an electrically switched liquid crystal layer.

According to a further example configuration, the reference structure RS realized in the contact glass KG acts in a non-visible spectral band and for example is realized by a dielectric reflection layer system.

By preference, this dielectric reflection layer system is designed as a bandpass filter such that it for example partially reflects the beams of an NIR target laser at a wavelength between 780 nm and 850 nm but predominantly transmits the treatment laser at a wavelength of 1064 nm and the visible light at wavelengths between 400 nm and 700 nm. However, at the OCDR wavelength, for example 1060 nm, the reference structure RZ realizes a backscatter of no more than 3%, for example <0.5%, in order to avoid saturation in the OCDR signal. For example, the reference structure RS is also designed such that, in the OCDR, it has a signal with a signal-to-noise ratio, especially in relation to the noise background caused by shot noise, of more than 10 dB, more than 20 dB or more than 30 dB, but also by preference of no more than 40 dB.

For example, the positions of target structures PZSₙ and of the reference structure PRS are identified by determining the maximum value or centroid value or a threshold value of the OCDR signal, or else by fitting a signal model. In this case, a position of a signal in the OCDR determined thus is for example assigned to a target or reference structure by using an expected signal sequence in the OCDR signal curve (for example, front or back side of the contact glass, corneal surface, optionally capsular bag front side, IOL front and back side, optionally capsular bag back side, retinal surface). In the process, characteristic signal strengths, for example at the contact glass or the IOL, can also serve for automated inclusion or exclusion of structures expected in the signal sequence. Characteristic signal curves can also be used to this end, for example sharp reflections at the IOL surfaces with at the same time a lower backscatter strength in the interior of the IOL, i.e. between the sharp surface reflections, in comparison with a crystalline lens. In this case, the characteristic signal curves can run axially, but also laterally. For example, capsular bag curves may have a substantially “wavier” profile laterally than the surfaces of conventional IOLs. Multifocal IOLs (e.g. Fresnel optics), for example, may even have typical, recognizable patterns. Furthermore, plausibility checks for possible or probable depth ranges of specific structures can be used, for example regarding probable corneal thickness, anterior chamber depth or else eye length ranges. Moreover, statements regarding the eye structure given by the operator can also be used, for example in the special case where phakic IOLs are used.

According to an example configuration, the imaging system is focused on the target structures ZS together with the laser beam of the laser treatment system, wherefore an autofocus system is used. By preference, an NA of the imaging system and an NA of the laser beam of the laser treatment system differ by a factor of <2.

Hereinbelow, the proposed method for recalibrating the focus of an ophthalmological system for intraocular laser treatment is described in more detail on the basis of a treatment system for laser vitreolysis.

The treatment system for laser vitreolysis also comprises an OCDR system in addition to a focusing unit and a control unit.

A sufficiently large pupil diameter, which depends on the working depth, must be ensured for laser vitreolysis. In detail, example pupil diameters are >4 mm for a laser vitreolysis treatment in the anterior region, >5 mm for such a treatment in the central region and >6 mm for such a treatment in the posterior region. For example, the verification of the pupil diameter can be used to ensure that the treatment laser can only be activated if a pupil diameter that is sufficiently large for the respective sought-after working depth was recognized.

In this respect, FIG. 1 shows the treatment system for laser vitreolysis 2 focused on a first target structure ZS₁ in the eye 1 to be treated.

The treatment system for laser vitreolysis 2 comprises a treatment laser 3, an OCDR system 4, an imaging system 5, an optical system 6 and a control unit (not depicted here).

The laser beam 7 of the treatment laser 3 is focused on a target structure ZS₁ in the eye 1 to be treated, by virtue of the distance A of the treatment system for laser vitreolysis 2 being changed in relation to the eye 1, represented by a reference structure RS (for example, the corneal surface), proceeding from a plane of reference RE, until, at an eye distance A=A₁, the focusing of the laser beam on the target structure ZS₁ (in this case the lens back side by way of example) is identifiable. In this case, focusing on ZS₁ can be identified by observing a maximized backscatter of the (attenuated) laser beam 7 or of a target laser (not depicted here) from the target structure ZS₁ by use of the imaging system 5 or, if focal positions of OCDR beam (not depicted here) and laser beam 7 are sufficiently matched to one another, by observing a maximized OCDR signal of the target structure (gray peak) at the position PZS₁ in the OCDR signal profile 8. Overall, the OCDR signal profile 8 extends over a relative depth range from 0 to Zₘₐₓ (optical path). In the case of focusing on the target structure ZS₁, the focal position PF₁ of the laser beam 7 in the OCDR then precisely corresponds to the OCDR position PZS₁ of the target structure.

For example, the front lens surface of the optical system 6 serves as further plane of reference BE here. Since the measurement range of the OCDR system 4 generally encompasses only slightly more than the overall length of the eye 1 to be treated, a reference plane RE at a distance ΔE from BE is set by way of the reference arm of the OCDR system 4, for the purpose of carrying out the measurements. For the focus recalibration, the assumption is made without loss of generality that the reference plane is not changed between the calibration steps. Should this nevertheless be the case, the positions PZSₙ should be adapted accordingly. In this context, it should be observed that the refractive index between BE and RE corresponds to that of the ambient medium (generally air with a refractive index of 1). The refractive index between RE and RS can be that of air, or it may include sections of glass or plastic refractive indices (for example, n=1.2 . . . 1.8) if a contact glass is used. Since, moreover, the refractive indices in the eye are slightly different (aqueous humor and vitreous humor approx. 1.36 and cornea approx. 1.38, IOL depending on material used) and may also vary from patient to patient, it is recommended in general to determine positions PZSₙ and also the focus position PF (ΔA) to be determined approximately as respective optical path lengths in relation to the reference plane RE. So as to have to make as few adjustments of ΔE as possible, or none at all, during the focus recalibration, the depth range 0 to Zₘₐₓ to be covered by the OCDR signal profile 8 is chosen so that at least the depth of the posterior eye portion (>25 mm optical path) is covered, but where possible also the entire average eye length (>34 mm optically) or else, ideally, an extended range of >60 mm or >100 mm (in each case optically). To this end, use should be made of OCDR systems with an appropriate coherence length.

Thereafter, the distance A₁ and the target structure position PZS₁ are determined from the OCDR signal profile 8, in each case as optical path lengths in relation to the reference plane RE. By way of example, in this case the front surface of the cornea is used as reference structure RS and the lens back side is used here as target structure ZS₁.

In the OCDR signal profile 8, the schematically depicted signal peaks, from left (0) to right (the maximum measurement range Zₘₐₓ), correspond to the front and back surface of the cornea, the front and back surface of the lens and the retinal surface of the eye 1 to be treated. For the sake of clarity, the background noise, signals from deeper retinal or choroidal layers and the capsular bag signals were not depicted here. Whether the capsular bag signals are distinguishable from lens surface signals depends on, for example, the specific situation, i.e. whether or not the capsular bag rests against the lens, and also on the sensitivity and resolution of the OCDR system.

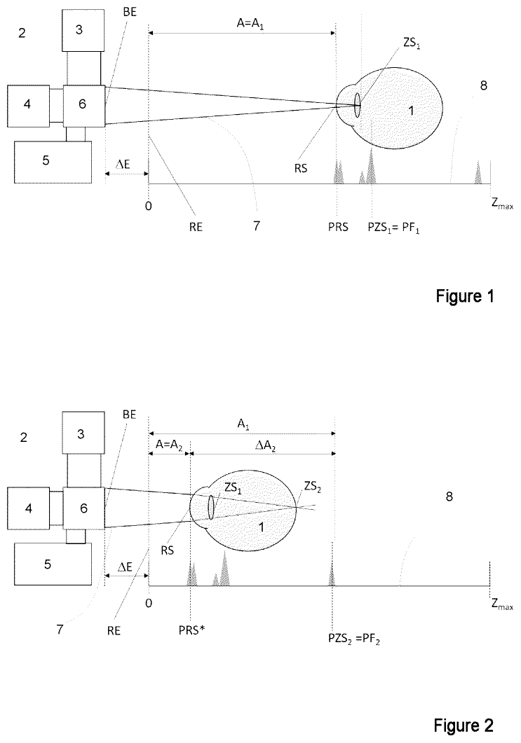

FIG. 2 shows the treatment system for laser vitreolysis 2 focused on a second target structure ZS₂ in the eye 1 to be treated.

To this end, the laser beam 7 of the treatment laser 3 is focused on a target structure ZS₂ in the eye 1 to be treated, by virtue of the distance A of the treatment system for laser vitreolysis 2 from the eye 1 being changed in such a way, proceeding from a plane of reference RE, in relation to the initial position A₁ (changed by ΔA) until the focusing of the laser beam on the respective target structure ZS₂ is identifiable again (as mentioned, for example based on maximized treatment or target laser backscatter or optionally local OCDR signal maximization in the case of a matched beam geometry between OCDR and treatment laser). To this end, the treatment system for laser vitreolysis 2 can be moved in relation to the eye 1 (by use of an equipment base (not depicted here) that is movable manually or by motor), or vice versa (for example by use of a motor-driven patient head support).

Subsequently, the distance A=A₂ or ΔA₂=A₁-A₂ and the position of the OCDR signal PZS₂ of the second target structure ZS₂ are determined from the OCDR signal profile 8, wherein the front surface of the cornea continues to serve as reference structure RS in this case and the retinal surface serves as second target structure ZS₂. The position of the reference structure in the OCDR signal profile 8 now is PRS*, which differs from the position of the reference structure PRS in the case of focusing on ZS₁.

On account of focusing on the second target structure ZS₂, the position of the target structure PZS₂ now also corresponds to the position PF₂ of the focus of the treatment laser 3, in each case as optical paths in relation to the reference plane RE again.

From the values A₁, ΔA₂, PZS₁, PZS₂ ascertained thus, it is now possible to derive at least one function

PF(ΔA)=f_{A}_{₁}_{,ΔA}_{₂}_{,PZS}_{₁}_{,PZS}_{₂}(ΔA)

which ideally yields

$$PF ( Δ {A}_{1} = 0 ) = {PZS}_{1} and PF ( Δ {A}_{2} ) = {PZS}_{2}$$

and with the aid of which it is possible to approximately determine the respective assumable focus position PF of the treatment laser 3 approximately for other values of a change in distance ΔA, selectable as desired. In this case, the function PF(ΔA) should where possible have a deviation between approximately determined focus position of the treatment laser and actual focus position of the treatment laser of less than 2 Rayleigh lengths of the treatment laser focus, for example less than 1 Rayleigh length or in another example less than 0.5 of the Rayleigh length.

The step of determining the parameters ΔA₂ and PZS₂ depicted in FIG. 2 can now be repeated analogously for further steps of focusing on target structures ZS₃, . . . , ZS_{N}, in order ultimately to obtain an extended set of parameters A₁, ΔA₂ . . . ΔA_{N}, PZS₁ . . . PZS_{N} which can then be used to determine a function PF (ΔA)=f_{A}_{₁}_{,ΔA}_{₂}_{. . . ΔA}_{_{N}}_{,PZS}_{₁}_{. . . PZS}_{_{N}}(ΔA) providing even greater accuracy for the estimate of the focus position of the treatment laser for any desired eye distances ΔA. In this case, too, the ascertained function should ideally run exactly through the measurement points, i.e. PF (ΔAₙ)=PZSₙ, but over the entire curve it should for example always enable a deviation between the approximately determined treatment laser focus position and actual treatment laser focus position of less than 2 Rayleigh lengths of the treatment laser focus, for example less than 1 Rayleigh length or in another example less than 0.5 of the Rayleigh length.

Since contact glasses are also used in laser vitreolysis treatment in particular, these will be discussed in more detail below.

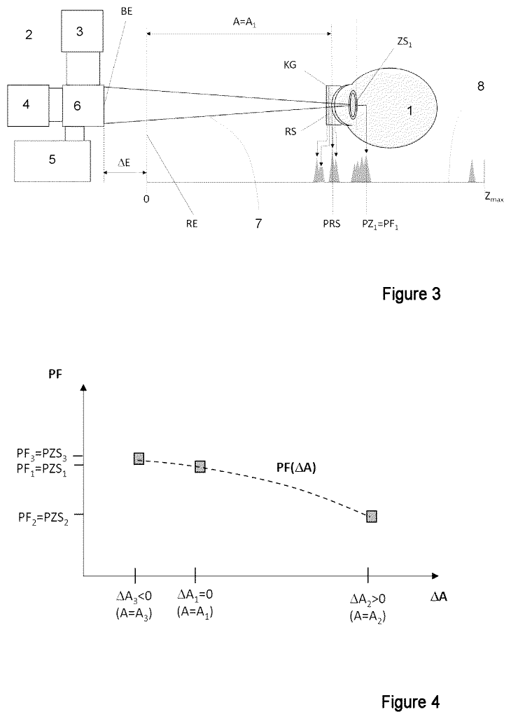

In this respect, FIG. 3 shows a treatment system for laser vitreolysis which uses a contact glass KG with a reference structure RS.

The treatment system for laser vitreolysis 2 comprises a treatment laser 3, an OCDR system 4, an imaging system 5, an optical system 6 and a control unit (not depicted here) and provides for the use of a contact glass KG.

The laser beam 7 of the treatment laser 3 is focused on a target structure ZS₁ (the capsular bag front side in this case) in the eye 1 to be treated, by virtue of the distance A of the treatment system for laser vitreolysis 2 being changed, proceeding from a plane of reference RE, in relation to the eye 1 until the focusing of the laser beam 7 on the respective target structure ZS₁ is identifiable (as mentioned, for example based on maximized treatment or target laser backscatter or optionally local OCDR signal maximization in the case of a matched beam geometry between OCDR and treatment laser).

Subsequently, the distance A=A₁ from the reference plane RE to the position of the reference structure PRS and also the target structure position PZS₁ are determined from the OCDR signal profile 8, in each case as optical path lengths in relation to the reference plane RE. In this case in the embodiment variant shown here, the reference structure RS is situated in the used contact glass KG. Thus, the reference structure is situated outside of the eye but has a sufficiently fixed relationship to the eye as a result of the contact.

On account of focusing on the target structure ZS₁, the position of the target structure PZS₁ thus also corresponds to the position PF₁ of the focus of the treatment laser 3.

In the OCDR signal profile 8, the depicted signal peaks from left (0) to right (the maximum measurement range Zₘₐₓ) correspond to the front surface of the contact glass, the technical structure RS in the contact glass, the front and back surface of the cornea, the front surface of the capsular bag, the front surface and back surface of the lens, the back side of the capsular bag and the retina of the eye 1 to be treated.

As described above, the front or back side of a contact glass KG present or a technical structure present in the contact glass, which may additionally be embodied to be modifiable on an individual basis, for example switchable or modulable, can be used as reference structure RS.

In FIG. 4, the function PF (ΔA) for determining any desired focus position PF of the laser beam of the laser treatment system is illustrated by way of example. In this case, the target laser beam of the laser treatment system was focused on three target structures ZS₁, ZS₂ and ZS₃ in the eye to be treated, by virtue of the distance A of the laser treatment system from the eye having been changed until the focusing of the target laser beam of the laser treatment system on the target structures ZS₁, ZS₂ and ZS₃ was detected and the corresponding distances A₁, A₂ and A₃ or changes in distance ΔA₂ and ΔA₃ in relation to A₁ were determined. Using the function PF (ΔA) arising from these sampling points, it is possible, for any selectable values of a change in distance ΔA of the laser treatment system from the eye, to determine the respective assumable focus position PF of the laser beam of the laser treatment system.

According to a further example configuration, the imaging system is moved together with the laser of the vitreolysis system and focusing on the target structures ZS is realized by use of an autofocus system in each case. In this case focusing is implemented by changing a focal length or changing a distance between system and patient's eye, for example, wherefore use can also be made, for example, of a motor-driven head support or a motor-driven equipment head.

In this context, it is advantageous if a numerical aperture (NA) of the imaging system and a numerical aperture of the treatment laser are similar, i.e. differ by a factor of <2.

It is also advantageous if the OCDR system and the treatment laser operate at similar wavelengths, i.e. with a deviation of <10%. In this case, use is for example made of wavelengths around 1060 nm since it is possible here to make use of long-coherent, tunable lasers for the OCDR (i.e. SS-OCDR), and YAG lasers at 1064 nm as treatment laser.

The proposed arrangement for recalibrating the focus of an ophthalmological system for intraocular laser treatment consists of a treatment laser unit, an imaging unit and an optical system for focusing and beam superposition, and also an OCDR system and a control unit.

Regarding the description of the function of the arrangement for recalibrating the focus of an ophthalmological system for intraocular laser treatment, reference is made to the above-described method.

According to the invention, the laser treatment system is designed such that the distance A from the eye is modifiable and a target laser beam is focusable on at least one target structure ZS₁ in the eye to be treated. The control unit is designed to determine a distance A₁ from the position of a chosen reference structure PRS, and the position of the target structure PZS₁ in the OCDR signal profile, in each case in relation to a reference plane RE, and, for any selectable values of a change in distance ΔA of the laser treatment system from the eye, to use the parameters A₁ and PZS₁ to determine a function

PF(ΔA)=f_{A}_{₁}_{,PZS}_{₁}(ΔA)

and to approximately calculate the respective assumable focus position PF of the laser beam of the laser treatment system in the OCDR signal profile.

For example, the laser treatment system is designed such that, in addition to ZS₁, the target laser beam is incrementally focusable on N-1 further target structures ZS₂, . . . , ZS_{N}. To this end, the control unit is designed to determine, in the OCDR signal profile, the respective positions of the target structures PZS₂, . . . , PZS_{N} and the respective changes ΔA₂, . . . , ΔA_{N} in the position of the reference structure vis-à-vis its initial position PRS=A₁ when focusing on the first target structure ZS₁ and then, using the parameters A₁, ΔA₂ . . . .ΔA_{N}, PZS₁ . . . . PZS_{N}, to determine a function PF (ΔA)=f_{A}_{₁}_{,ΔA}_{₂}_{. . . ΔA}_{_{N}}_{, PZS}_{₁}_{. . . PZS}_{_{N}}(ΔA), from which, for any selectable values of a change in distance ΔA of the laser treatment system from the eye, the respective assumable focus position PF of the laser beam of the laser treatment system in the OCDR signal profile is approximately calculable.

A first group of example configurations relates to the laser treatment system. Thus, for example it is possible that the treatment laser beam of the laser treatment system with a reduced pulse energy, which cannot trigger photodisruption, is usable as target laser beam. However, it is also possible that an additional laser beam whose parameters do not allow permanent tissue change in the eye is used as target laser beam.

Further, provision can be made for the laser treatment system to comprise a plurality of target lasers which intersect at the location of the focus of the treatment laser of the laser treatment system and which serve to focus on a target structure ZS in the eye to be treated. In this case, the continuous wave laser beams of the target laser for example have an identical or similar focal position as the laser beam of the laser treatment system.

According to a further example configuration, the laser treatment system itself is designed to create target structures ZS in the eye. For example, this can be implemented by a pulse or a modulation of the target laser which bring about a modification in the eye and bring about a signal change in the OCDR or a modified backscatter or a phase or speckle grain modification in the OCDR signal. These target structures ZS are temporary or modifiable, are created by at least one laser shot for example and bring about the formation of a gas bubble or a speckle structure in the OCDR that has been temporarily modified as a consequence of temperature changes.

According to another example configuration, the laser treatment system is designed for laser vitreolysis and, by way of a laser beam, creates a target structure ZS in the vicinity of a structure to be worked on.

A second group of example configurations relates to the control unit, which is designed to establish the detection of focusing on a target structure by automated establishment of one or more of the following states:
maximized backscatter of the target laser from the target structure,
minimized diameter of the target laser beam light distribution on the target structure and/or
characteristic state or characteristic change in the OCDR signal of the target structure.

The control unit is also designed to use the target structure ZS created by a laser shot of the laser beam of the laser treatment system not only to determine the focal positions but also to titrate the laser power.

Moreover, the positions of target structures and of the reference structure can be identified by the control unit, for example by determining the maximum value or the centroid value or a threshold value of an OCDR signal in the OCDR signal profile.

The control unit uses a polynomial of first to N-th degree or a different non-linear function with N degrees of freedom for the function f_{A}_{₁}_{, A}_{₂}_{. . . ΔA}_{_{N}}_{, PZS}_{₁}_{. . . PZS}_{_{N}}(ΔA) for determining the focus positions PF.

However, it is also possible to use a polynomial or non-linear function of a degree greater than N. To determine the function f, it is possible to additionally consider parameters of the contact glass, for example radii of curvature, thicknesses or refractive indices, ascertained otherwise or additional parameters of the eye, for example refractive indices, thicknesses or radii of cornea or lens, ascertained otherwise.

For example, the control unit is designed such that the deviation of the approximate determination of the assumable focus position PF of the laser beam is less than 2 Rayleigh lengths of the laser focus, for example less than 1 Rayleigh length or in another example less than 0.5 of the Rayleigh length.

A third group of example configurations relates to an additionally present camera system, which captures the target lasers and the target structure ZS. The control unit uses these recordings to determine the focusing, for example by
minimizing the spacing of target beam laser positions,
minimizing the spatial variation of a periodically moving target laser beam, or
maximizing the backscatter from at least one target beam laser focus.

A fourth group of example configurations relates to an additionally used contact glass, the front or back side thereof, or a technical structure situated in the contact glass, serving as reference structure RS.

In this case the technical structure realized in the contact glass KG as reference structure RS creates a characteristic signal in the OCDR, having a specific level, plateau, curve, position, distance or multiple peaks, or a characteristic polarization dependence of the signal. By preference, the realized reference structure RS is modifiable, for example switchable or modulable, for example by way of a modification of the scattering or polarization.

The reference structure RS realized in the contact glass KG acts in a non-visible spectral band and for example is realized by a dielectric reflection layer system. For example, the dielectric reflection layer system is designed in such a way that the target laser beams at a wavelength of between 400 nm . . . 1050 nm are reflected and the treatment laser beam of the laser treatment system at wavelengths >1050 nm is predominantly transmitted.

A fifth group of example configurations relates to an imaging system present, which, for example by way of an autofocus system, is focused on the target structures ZS together with the laser beam of the laser treatment system.

For example, an NA of the imaging system and an NA of the laser beam of the laser treatment system differ by a factor of <2.

However, the imaging system and the laser beam of the laser treatment system may also have a certain focusing difference in order to compensate for different wavelengths.

By preference, the imaging system is designed to ensure, depending on the working depth, a sufficiently large pupil diameter.

According to an example configuration, the imaging system is designed to depict, in relation to the eye structures prior to the treatment laser activation, the focal position estimated by way of the focus recalibration.

A last group of example configurations relates to the OCDR system.

By preference, an NA of the OCDR system and an NA of the laser beam of the laser treatment system differ by a factor of <2.

For example, the wavelengths of the OCDR system and of the laser beam of the laser treatment system deviate by no more than 10% from one another.

However, it is also possible that the OCDR system and the laser beam of the laser treatment system have a certain focusing difference in order to compensate for different wavelengths.

The method according to the invention and the arrangement provide a solution for recalibrating the focus of an ophthalmological laser treatment system which rectifies the disadvantages of the known technical solutions and takes account of the individuality of an eye to be treated and the tolerances of the optical system when recalibrating the focus of the treatment laser.

As a result, it is possible to determine the focal position of the treatment laser for each new treatment situation, to be precise independently of the change of a patient and/or contact glass, or else a modification of the focal length of the treatment laser, of the accommodation and/or for example of the position of the patient's eye.

Moreover, the proposed solution is easy to implement, cost effective and enables a simpler, faster and, above all, safer laser treatment on the eye.

By way of the solution according to the invention, it is possible to reliably and precisely set up blocked regions, within which a laser treatment can be precluded; this is useful for systems for laser vitreolysis, in order to protect and spare sensitive eye structures.

Even though the proposed solution is provided for treatment systems for laser vitreolysis in particular, it offers numerous other intraocular application options, as mentioned above, which would also profit from the proposed solution for recalibrating the focus of a laser treatment system.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112 (f) are not to be invoked unless the specific terms “means for” or “step for” are recited in a claim.

1-72. (canceled)

73. A method to recalibrate a focus of an ophthalmological system for intraocular laser treatment, the ophthalmological system also comprising an OCDR system and a control unit in addition to a treatment laser unit, an imaging unit and an optical system that performs focusing and beam superposition, the method comprising:
focusing a target laser beam of the system for ophthalmological laser treatment on at least one target structure ZS₁ in an eye to be treated by adjusting a distance A of the ophthalmological system for laser treatment from the eye being treated until focusing of the target laser beam of the laser treatment system on the target structure ZS₁ is detected;
determining a distance A₁ from a position of a chosen reference structure PRS, and a position of a target structure PZS₁ in an OCDR signal profile, in each case in relation to a reference plane RE, and wherein, for any selectable values of a change in distance ΔA of the laser treatment system from the eye, a respective assumable focus position PF of the laser beam of the laser treatment system in the OCDR signal profile is estimated approximately using the parameters A₁ and PZS₁.

74. The method as claimed in claim 73, further comprising using the ascertained values A₁ and PZS₁ to determine a function
PF(ΔA)=f_{A}_{₁}_{,PZS}_{₁}(ΔA)
which, for any selectable values of a change in distance ΔA of the laser treatment system from the eye, approximately calculates the respective assumable focus position PF of the laser beam of the laser treatment system in the OCDR signal profile.

75. The method as claimed in claim 73, wherein, apart from ZS₁, the target laser beam is incrementally focused on N-1 further target structures ZS₂, . . . , ZS_{N} and, to this end, the respective positions of the target structures PZS₂, PZS_{N} and the respective changes ΔA₂, . . . , ΔA_{N} in the position of the reference structure vis-à-vis its initial position PRS=A₁ when focusing on the first target structure ZS₁ are determined in the OCDR signal profile, and wherein the parameters A₁, ΔA₂ . . . ΔA_{N}, PZS₁ . . . . PZS_{N} are then used to determine a function PF (ΔA)=f_{A}_{₁}_{,ΔA}_{₂}_{. . . ΔA}_{_{N}}_{, PZS}_{₁}_{. . . PZS}_{_{N}}(ΔA), which, for any selectable values of a change in distance ΔA of the laser treatment system from the eye, approximately calculates a respective assumable focus position PF of the laser beam of the laser treatment system in the OCDR signal profile.

76. The method as claimed in claim 73, further comprising implementing the detection of focusing on a target structure by visual or automated establishment of one or more of the following states: 1) maximized backscatter of the target laser from the target structure, 2) minimized diameter of the target laser beam light distribution on the target structure, 3) characteristic state or characteristic change in the OCDR signal of the target structure.

77. The method as claimed in claim 73, further comprising focusing target lasers on the target structure ZS by virtue of the spatial variation of a periodically moving target laser beam being minimized.

78. The method as claimed in claim 73, further comprising implementing the focusing of at least one target laser on the target structure ZS by maximizing backscatter from at least one target beam laser focus via confocal detection.

79. The method as claimed in claim 77, further comprising using a continuous wave laser beam with the same or similar focal position as the laser beam of the laser treatment system as target laser.

80. The method as claimed in claim 73, further comprising using a lens back side, a capsular bag back side, a retinal surface or other structures in the eye as target structures ZS.

81. The method as claimed in claim 76, further comprising creating a target structure ZS in the eye by a pulse or a modulation of a laser beam of the laser treatment system, said target structure being a modification in the vitreous humor or any other eye structure, which brings about a signal change in the OCDR, or a modified backscatter or a phase or speckle grain modification in the OCDR signal.

82. The method as claimed in claim 77, further comprising creating a target structure ZS in the eye by a pulse or a modulation of a laser beam of the laser treatment system, said target structure being a modification in the vitreous humor or any other eye structure, which brings about a signal change in the OCDR, or a modified backscatter or a phase or speckle grain modification in the OCDR signal.

83. The method as claimed in claim 73, further comprising using a front or a back side of a contact glass KG present, a technical structure situated in the contact glass, or eye structures including a front or a back side of cornea, a front or a back side of lens or a front or a back side of capsular bag or a retinal surface serve as reference structure RS.

84. The method as claimed in claim 73, wherein the technical structure in the contact glass KG as reference structure RS is configured to create a characteristic signal in the OCDR, having a specific level, plateau, curve, position, distance or multiple peaks, or a characteristic polarization dependence of the signal.

85. The method as claimed in claim 73, wherein the reference structure RS in the contact glass KG is modifiable, including switchable or modulable, by way of a modification of the scattering or polarization.

86. The method as claimed in claim 83, wherein the reference structure RS in the contact glass KG acts in a non-visible spectral band or comprises a dielectric reflection layer system.

87. The method as claimed in claim 86, wherein the dielectric reflection layer system acts in such a way that the target laser beams at a wavelength of between 400 nm and 1050 nm are reflected and the treatment laser beam of the laser treatment system at wavelengths >1050 nm is predominantly transmitted.

88. The method as claimed in claim 73, wherein identification of the positions of the target structures and of the reference structure is implemented by determining the maximum value or the centroid value or a threshold value of an OCDR signal in the OCDR signal profile.

89. The method as claimed in claim 75, further comprising using a function f_{A}_{₁}_{,ΔA}_{₂}_{. . . ΔA}_{_{N}}_{, PZS}_{₁}_{. . . PZS}_{_{N}}(ΔA) that determines the focus positions PF is a polynomial of first to N-th degree or a different non-linear function with N degrees of freedom.

90. The method as claimed in claim 73, further comprising using a function f_{A}_{₁}_{,ΔA}_{₂}_{. . . ΔA}_{_{N}}_{, PZS}_{₁}_{. . . PZS}_{_{N}}(ΔA) that determines the focus positions PF that is a polynomial or a non-linear function of a degree greater than N and additional parameters of the contact glass ascertained otherwise, including at least one of radii of curvature, thicknesses or refractive indices, to determine the function f.

91. The method as claimed in claim 73, further comprising using a function f_{A}_{₁}_{,ΔA}_{₂}_{. . . ΔA}_{_{N}}_{, PZS}_{₁}_{. . . PZS}_{_{N}}(ΔA) that determines the focus positions PF is a polynomial or a non-linear function of a degree greater than N and additional parameters of the eye ascertained otherwise, including at least one of refractive indices, thicknesses or radii of cornea or lens, to determine the function f.

92. The method as claimed in claim 75, wherein the target structures ZS₁, . . . , ZS_{N} each contain at least one structure in front and back vitreous humor regions, with by preference the front or back side of the cornea and the retinal surface are selected.

93. The method as claimed in claim 75, further comprising also choosing target structures ZSₙ located in desired exclusion zones for treatment of laser vitreolysis, selected from a group including at least the lens back side and the retinal surface.

94. The method as claimed in claim 93, further comprising creating the target structure ZS by a laser beam of the treatment system for laser vitreolysis in a vicinity of a structure to be worked on.

95. The method as claimed in claim 86, further comprising enabling a pupil diameter >4 mm for a laser vitreolysis treatment in an anterior region, enabling a pupil diameter >5 mm for a laser vitreolysis treatment in a central region, and enabling a pupil diameter >6 mm for a laser vitreolysis treatment in a posterior region.
